SEQUENCE LISTING (2 sequences)

SEQ ID NO: 1 - DNA, 20 bp, Artificial Sequence
  Synthesized
aacagtcttgtacaagtcca

SEQ ID NO: 2 - DNA, 21 bp, Artificial Sequence
  Synthesized
ggtgcttttgatatttttccg

(19) United States
(12) Patent Application Publication
(10) Pub. No.: US 2020/0340040 A1
(43) Pub. Date: Oct. 29, 2020

(54) METHOD FOR QUANTITATIVELY DETECTING VBNC STATE BACTERIA

(71) Applicant: CHINA AGRICULTURAL UNIVERSITY, Beijing (CN)

(72) Inventors: Xiaojun LIAO, Beijing (CN); Kai DONG, Beijing (CN); Hanxu PAN, Beijing (CN); Yongtao WANG, Beijing (CN)

(21) Appl. No.: 16/963,144

(22) PCT Filed: Jan. 19, 2018
(86) PCT No.: PCT/CN2018/073327

§ 371 Date: Jul. 17, 2020

(51) Int. Cl. C12Q 1/686; G01N 1/28; C12Q 1/689

(52) U.S. Cl. CPC C12Q 1/686; C12Q 2600/158; C12Q 1/689; G01N 1/28

(57) ABSTRACT

The present invention discloses a method for quantitatively detecting VBNC state bacteria. The method of the present invention comprises the following steps: treating HPCD-induced VBNC state E. coli O157:H7 with PMA to eliminate the impact of dead and damaged bacteria in the sample on quantification; using the genomic DNA of PMA-treated VBNC state bacteria as a template, ddPCR was performed. The present invention establishes a PMA-ddPCR detection method for rapid quantitative detection of the number of VBNC state bacteria. The detection method of the present invention can achieve accurate detection and quantification of VBNC state bacteria within 4-6 h with a detection range of 10¹-10⁷ and a quantitative range of 10²-10⁷. This method not only has the advantages of strong specificity and high sensitivity, but also has the advantages of accurate quantification, reliable results, simplicity and time saving. The present invention is of great significance both for the detection and quantification of VBNC state bacteria in food and for the management and monitoring of food safety.

### RELATED APPLICATIONS

The present application is a National Phase of International Application Number PCT/CN2018/073327, filed Jan. 19, 2018.

### INCORPORATION BY REFERENCE

The sequence listing provided in the file entitled Sequence_Listing_2020-07-15.txt, which is an ASCII text file that was created on July 15, 2020, and which comprises 566 bytes, is hereby incorporated by reference in its entirety.

### TECHNICAL FIELD

The present invention belongs to the technical field of food safety and biological detection, and relates to a method for quantitatively detecting VBNC state bacteria in food, and particularly to a method for quantitatively detecting VBNC state Escherichia. coli O157:H7.

### BACKGROUND ART

In the unfavorable environment of the outside world, many bacteria will enter a viable but nonculturable (VBNC) state. This state is a dormant form of non-spore-forming bacteria, which can improve the ability of bacteria to survive in unfavorable environments. At present, it is known that more than 80 kinds of bacteria can enter a viable but nonculturable state, most of which are pathogenic bacteria. Although VBNC bacteria still have metabolic activity, they cannot grow or form colonies on the non-selective media commonly used by the bacteria. Conventional bacterial detection methods such as plate count method cannot detect the presence of VBNC bacteria, which may underestimate the number of bacteria in the test sample and bring safety risks to people. Therefore, the development of detection methods for VBNC state bacteria is essential for the effective killing of VBNC bacteria.

The criterion for judging the viable but nonculturable state is that the number of culturable bacteria is zero but the number of viable bacteria is not zero. The determination of the number of viable bacteria is the key to determine whether the unculturable bacteria are dead or enter the VBNC state. At present, the most common detection method for VBNC state are as follows: \textcircled{1} detecting the integrity of cell structure (such as cell membrane); this method relies on fluorescent dyes to distinguish dead and live bacteria, mainly using the characteristic that some fluorescent dyes have different permeabilities to the cell membrane; some fluorescent dyes, such as SYTO9, SYBR-Green I, can penetrate intact and damaged cell membranes, while some fluorescent dyes, such as EB, PI, can only pass through damaged cell membranes; dead and live bacteria can be distinguished by combining dyes with different cell membrane permeabilities, and then the number of live bacteria can be obtained by using a flow cytometer; currently the most commonly used is the Live/Dead Baclight kit; \textcircled{2} detecting the expression of specific genes in VBNC bacteria by PMA combined with RT-PCR (Real-time PCR); propidium monoazide (PMA) is a high-affinity photoreactive DNA-binding dye that can enter the cell through a damaged cell membrane and irreversibly covalently bind to DNA to prevent the DNA of dead or damaged cells from being amplified. Therefore, the bacteria that can be amplified are considered to be VBNC bacteria. However, the above methods have certain defects. The flow cytometer counts VBNC bacteria by defining the percentage of VBNC in the treatment sample group by comparing the distribution areas of live bacteria and completely dead bacteria on the flow cytometer data chart and this method can only obtain a rough percentage of VBNC bacteria; while the main shortcoming of the method of PMA combined with RT-PCR is that the premise of successful RT-PCR experiment is to determine the amplification efficiency of primers, and largely depends on the Ct value, and thus this method has poor repeatability and is easy to cause experimental errors, so it is difficult to achieve accurate quantification.

With the continuous updating of PCR instruments, droplet digital PCR (ddPCR) has become a rapid and accurate PCR technology that can realize absolute quantification of DNA in recent years. The principle is to distribute DNA molecules diluted to a certain concentration in a certain number of droplets, so that the number of DNA molecules in most droplets is 1 or 0, and then the number of positive droplets is determined by PCR amplification and cumulative reading of fluorescent signals, and then the number of DNA molecules in the sample is calculated according to the poisson distribution. The quantitative method of digital PCR no longer depends on the cycle threshold of the amplification curve, so it is very little affected by the amplification efficiency, and does not need to use internal reference and standard curve. This method has good repeatability and accuracy, and can achieve absolute quantification analysis of samples. At present, ddPCR has been applied to the detection of salmonella, E. coli O157:H7, Listeria monocytogenes, Enterobacter sakazakii, Staphylococcus aureus and other food-borne pathogens. However, there is a big problem in the detection of method. After the bacteria are induced into the VBNC state, there are not only VBNC bacteria that are still active, but also dead or damaged bacteria in the system and after the genome was extracted and amplified, the dead/live bacteria cannot be distinguished.

### SUMMARY OF THE INVENTION

The first object of the present invention is to provide a method for quantitatively detecting VBNC state bacteria.

The method for quantitatively detecting VBNC state bacteria provided by the present invention comprises the following steps:

1) treating the VBNC state bacteria to be tested with propidium monoazide to obtain propidium monoazide-treated bacteria;

2) performing ddPCR amplification on a target gene in the VBNC state bacteria to be tested with the genomic DNA of the propidium monoazide-treated bacteria as a template to obtain a copy number of the target gene;

3) determining the number of the VBNC state bacteria to be tested according to the copy number of the target gene.

In the above method, the method for treating the VBNC state bacteria to be tested with propidium monoazide comprises the following steps: mixing a bacteria solution of the VBNC state bacteria to be tested with propidium monoazide and incubating the resulting mixture to obtain an incubation product; subjecting the incubation product to light treatment to obtain the propidium monoazide-treated bacteria.

Because propidium monoazide (PMA) can bind to the DNA of dead or damaged bacteria, and the DNA is irreversibly modified, so that it cannot be amplified. However, propidium monoazide cannot enter the bacteria with intact cell membranes, which means that the genomic DNA of the VBNC state bacteria can be amplified normally. The present invention uses propidium monoazide to treat VBNC state bacteria to be tested or samples to be tested to distinguish VBNC state bacteria from dead or damaged bacteria, and then realize absolute quantitative counting of the VBNC state bacteria by ddPCR.

In the above methods, the ratio of the VBNC state bacteria to be tested to propidium monoazide is 1×10⁷ CFU:(15-23) μg. Preferably, the ratio of the VBNC state bacteria to be tested to propidium monoazide is 1×10⁷ CFU:20 μg.

In the above methods, the incubation condition is 30° C. for 15-30 min. Specifically, the incubation condition is 30° C. for 30 min.

In the above methods, the light treatment is illuminating the incubation product at a distance of 20 cm from a 500 W halogen lamp for 10-20 min. Specifically, the light treatment is illuminating the incubation product at a distance of 20 cm from a 500 W halogen lamp for 15 min.

In the above methods, the bacteria can be any bacteria in the prior art, such as Escherichia coli, Vibrio cholerae, Helicobacter pylori, Mycobacterium tuberculosis, Salmonella typhimurium, Listeria monocytogenes, etc. Specifically, the bacteria are E. coli strains. In the present invention, the E. coli strains are E. coli O157:H7 strains.

In the above methods, the target gene can be rfbe gene. The rfbe gene is a single copy gene in E. coli, so the copy number of the rfbe gene is directly equal to the number of bacterial cells, and the number of bacterial cells can be calculated based on the copy number of the rfbe gene. In practical applications, when detecting VBNC state E. coli O157:H7, or detecting other VBNC state E. coli or bacteria, other target genes can be selected for ddPCR amplification. Preferably, single copy target genes are selected and according to the copy number of the target gene, the number of bacterial cells can be directly calculated.

In the above methods, the primer pair used for the ddPCR amplification consists of the single-stranded DNA molecule set forth in SEQ ID NO: 1 and the single-stranded DNA molecule set forth in SEQ ID NO: 2.

In the above methods, the final concentration of each primer in the primer pair in ddPCR amplification reaction system is 500 nmol/L; the annealing temperature of the ddPCR amplification is 60° C. Specifically, the ddPCR reaction system is as follows: 10 μl of 2×PCR mixed solution (Bio-Rad), 1 μl of forward primer set forth in SEQ ID NO: 1, 1μl of reverse primer set forth in SEQ ID NO: 2, 1 μl of DNA template, 7 μl of H₂O. The ddPCR reaction procedure is as follows: 95° C. for 5 min; 40 cycles of (95° C. for 30 s, 60° C. for 60 s); 4° C. for 5 min; 95° C. for 10 min, rise/fall rates of temperature are 2.0° C./s.

The second object of the present invention is to provide new uses of the above methods.

The present invention provides use of the above methods for quantitative detection of VBNC state bacteria in a sample to be tested.

The present invention provides use of the above methods for quantitative detection of live bacteria in a sample to be tested.

The third object of the present invention is to provide a method for quantitatively detecting VBNC state bacteria in a sample to be tested.

The method for quantitatively detecting VBNC state bacteria in a sample to be tested provided by the present invention comprises the following steps:

1) treating the sample to be tested with propidium monoazide to obtain a propidium monoazide-treated sample;

2) performing ddPCR amplification on a target gene in the VBNC state bacteria in the sample to be tested with the genomic DNA of the propidium monoazide-treated sample as a template to obtain a copy number of the target gene;

3) determining the number of the VBNC state bacteria in the sample to be tested according to the copy number of the target gene.

In the above method, the method for treating the sample to be tested with propidium monoazide comprises the following steps: mixing the sample to be tested with propidium monoazide and incubating the resulting mixture to obtain an incubation product; subjecting the incubation product to light treatment to obtain the propidium monoazide-treated sample.

In the above methods, the incubation condition is 30° C. for 15-30 min. Specifically, the incubation condition is 30° C. for 30 min.

In the above methods, the light treatment is illuminating the incubation product at a distance of 20 cm from a 500 W halogen lamp for 10-20 min. Specifically, the light treatment is illuminating the incubation product at a distance of 20 cm from a 500 W halogen lamp for 15 min.

In the above methods, the sample to be tested contains VBNC state bacteria and they can be food processed by physical and/or chemical means such as low temperature and drying, or other samples containing VBNC state bacteria. The bacteria can be any bacteria in the prior art, such as Escherichia coli, Vibrio cholerae, Helicobacter pylori, Mycobacterium tuberculosis, Salmonella typhimurium, Listeria monocytogenes, etc. In practical applications, the corresponding target genes can be selected according to the bacteria that need to be tested, and ddPCR is performed on the genomic DNA of the bacteria in the sample to be tested with primers for amplifying the target gene to obtain a copy number of the target gene, and then the number of the bacteria in the sample to be tested can be determined according to the copy number of the target gene.

The last object of the present invention is to provide a kit for quantitative detection of VBNC state bacteria.

The kit provided by the present invention comprises propidium monoazide and a primer pair used for ddPCR amplification of a target gene in the bacteria.

In the above kit, the bacteria are E. coli strains and in the present invention, the E. coli strains are E. coli O157:H7 strains.

In the above kits, the target gene can be rfbe gene.

In the above kits, the primer pair used for ddPCR amplification of the rfbe gene consists of the single-stranded DNA molecule set forth in SEQ ID NO: 1 and the single-stranded DNA molecule set forth in SEQ ID NO: 2.

### DESCRIPTION OF THE DRAWINGS

FIG. 1 shows the proportion of VBNC bacteria in the PMA-ddPCR detection system.

FIG. 2 shows the proportion of VBNC bacteria in the flow cytometry analysis system.

FIG. 3 shows the effect of different concentrations of PMA on the genome amplification of dead bacteria.

FIG. 4 shows PMA-ddPCR detection of live bacteria in a mixed system of live bacteria and dead bacteria.

FIG. 5 is a comparison of the results of ddPCR detection of gradient diluted live bacteria and plate count method and their correlation analysis.

FIG. 6 shows the detection of sensitivity of ddPCR.

FIG. 7 shows the detection of specificity of ddPCR.

### DETAILED DESCRIPTION OF THE INVENTION

Unless otherwise specified, the experimental methods used in the following examples are conventional methods.

Unless otherwise specified, the materials and reagents used in the following examples are commercially available.

The strain E. coli O157:H7 (NCTC12900) used in the following examples belongs to E. coli O157:H7 EDL933, cannot produce shiga toxins stx1 and stx2, and belongs to the detoxified strain, which is from the British National Collection of Type Cultures.

The LB liquid medium used in the following examples consists of a solvent and solutes, wherein the solutes and their concentrations in the medium are as follows: tryptone 10 g/L, yeast extract 5 g/L and sodium chloride 10 g/L, pH is adjusted to 7.4 with NaOH.

The LB solid medium used in the following examples consists of a solvent and solutes, wherein the solutes and their concentrations in the medium are as follows: tryptone 10 g/L, yeast extract 5 g/L, sodium chloride 10 g/L and agar powder 15 g/L.

### EXAMPLE 1

### Quantitative Detection Method for VBNC State Bacteria and Optimization of Detection Conditions

I. Quantitative Detection Method for VBNC State Bacteria

1. Activation and Preparation of E. coli O157: H7

Strain E. coli O157:H7 (NCTC12900) at −80° C. was streaked on a solid LB Petri dish and incubated overnight in a 37° C. incubator (approximately 16-18 h), then single colonies were picked and inoculated into liquid LB medium, incubated at 200 rpm on a 37° C. shaker overnight (approximately 10-12 h), then transferred to fresh liquid LB medium at a ratio of 1:100, and incubated at 200 rpm on a 37° C. shaker for 2-3 h until an OD600=0.8 was reached, and finally the bacteria were collected and suspended in 0.85% (mass fraction) NaCl aqueous solution to obtain a bacteria solution to be induced.

2. Induction of VBNC State E. coli O157: H7

With reference to the Chinese Patent No. 102899272 B, a dense phase carbon dioxide device (model CAU-HPCD-1, disclosed in patent ZL200520132590.X) was used to induce the bacteria solution to be induced to a VBNC state to obtain VBNC state E. coli O157:H7. The specific steps were as follows: 20 mL of the bacteria solution to be induced (bacterial suspension) was filled into a glass bottle and the glass bottle was sealed with a parafilm; then the bacteria solution was placed in a reaction kettle and subjected to HPCD treatment (treatment pressure: 5 MPa, treatment temperature: 25° C.; pressure-holding time: 40 min). After the above treatment parameters were reached, the pressure was immediately released to obtain an induced bacteria solution.

The culturability of bacteria in the induced bacteria solution was detected using plate count method. The specific steps were as follows: 1 mL of HPCD-treated bacteria solution (induced bacteria solution) was detected by pouring plate method, cultured upside down in a 37° C. incubator for 24 h and then colonies were counted. The results showed that there were no colonies growing on the plate.

3. PMA Pretreatment of VBNC State E. coli O157:H7

1 ml of the induced bacteria solution (VBNC state E. coli O157:H7) was subjected to a 1:10 stepwise dilution to obtain a bacteria solution with a concentration of 10⁷ CFU/ml, and then 1 ml of the bacteria solution with a concentration of 10⁷ CFU/ml was taken, and 20 μg PMA (US EVERBRIGHT® INC., product number: P-4051) was added, incubated at 30° C. in the dark for 30 min, and the incubation product was illuminated at a distance of 20 cm from a 500 W halogen lamp for 15 min to make the PMA fully react and obtain a PMA-treated bacteria solution.

PMA can bind to the DNA of dead or damaged bacteria, and the DNA is irreversibly modified, so that it cannot be amplified. However, PMA cannot enter the bacteria with intact cell membranes, which means that the genomic DNA of the VBNC state bacteria can be amplified normally.

4. Genome Extraction of VBNC State E. coli O157: H7 After PMA Treatment

The total bacterial genomic DNA after PMA treatment was extracted with Tiangen bacterial genomic kit extraction kit (TIANGEN BIOTECH (BEIJING) CO., LTD.), eluted with 50 μL of TE solution, and the quality of genomic DNA was detected by Bioteke ND5000 and agarose gel electrophoresis.

5. ddPCR Detection of Number of Bacteria Entering VBNC State in Total Bacteria 1) Primer Design

The rfbe gene encodes E. coli O157:H7 0 antigen-specific synthetase, and participates in the biosynthesis of 0 antigen lipopolysaccharide. It is the basis for identifying E. coli O157:H7. The rfbE gene was used as a target and rfbE-specific primers were designed. The size of the amplified fragment was 80-200 bp. The primer sequences were as follows: forward primer rfbE-F for specific detection of the target gene rfbE of E. coli: 5′-AACAGTCTTGTACAAGTCCA-3′ (SEQ ID NO: 1); reverse primer rfbE-R for specific detection of the target gene rfbE of E. coli:

(SEQ ID NO: 2)
5′-GGTGCTTTTGATATTTTTCCG-3′.

2) ddPCR

Using bacterial genomic DNA as a template, ddPCR was performed with rfbE-F and rfbE-R.

The ddPCR reaction system was as follows: 10 μl of 2×PCR mixed solution (Bio-Rad), 1 μl of forward primer rfbE-F, 1 μl of reverse primer rfbE-F, 1 μl of DNA template, 7 μl of H₂O. The final concentrations of the forward primer rfbE-F and the reverse primer rfbE-F in the reaction system were both 500 nmol/L.

Droplets were prepared using BioRad's droplet generator. The prepared droplets were transferred to a 96-well plate and amplified on a PCR instrument according to the following procedure: 95° C. for 5 min; 40 cycles of (95° C. for 30 s, 60° C. for 60 s); 4° C. for 5 min; 95° C. for 10 min, rise/fall rates of temperature were 2.0° C./s.

3) Calculation of Number of VBNC Bacteria According to Copy Number of rfbe Gene in ddPCR Result

The 96-well plate was placed in a droplet analyzer, and the droplets of each sample were sequentially pipetted and passed through a two-color detector one by one with a carrier liquid flow. The droplets with a fluorescent signal were positive, and the droplets without any fluorescent signal were negative. The software recorded the proportion of positive droplets in each sample, and the data were automatically analyzed using Quantsoft2.0 software for digital PCR and the copy number of the rfbe gene in the sample to be tested was calculated according to the poisson distribution. The rfbe gene is a single copy gene in E. coli, so the copy number of the rfbe gene is directly equal to the number of bacterial cells, and then the number of VBNC state bacteria can be calculated.

The results of PMA-ddPCR detection of the number of VBNC bacteria are shown in FIG. 1. It was shown that the copy number of the rfbe gene was 350 copies/μl and the copy number of the rfbe gene of total bacteria was 7190 copies/μl. Therefore, the quantitative proportion of VBNC bacteria was 350/7190=4.87%.

4) Verification of Detection Results of ddPCR

The proportion of the VBNC state bacteria in 1 mL of HPCD-treated bacteria (induced bacteria solution) in step 2 was analyzed on a BD-C6 flow cytometer using PI/SYTO 9 double staining method by the Live/Dead BacLight Bacterial Viability assay kit (Invitrogen) and the degree of agreement between the detection results of ddPCR and the analysis results of flow cytometry by staining were analyzed. The specific steps for determining the number of live bacteria using the PI/SYTO 9 double staining method were as follows: the ready dye mixture (volume ratio of PI to SYTO 9 was 1:1) (Thermo Fisher Scientific) was mixed with the induced bacteria solution at a ratio of 3:1000 and after mixed evenly, the mixture was incubated at room temperature for 15 min in the dark; after incubation, the mixture was analyzed on a BD flow cytometer.

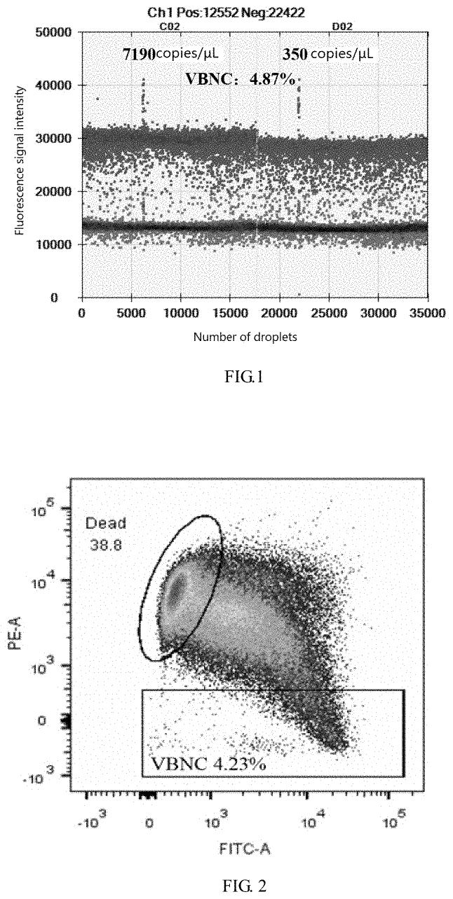

The results of flow cytometer are shown in FIG. 2, wherein the number of SYTO9 positive and PI negative bacteria was 4.23%, i.e., the number of VBNC bacteria was 4.23%, which was basically consistent with the detection results of PMA-ddPCR. It shows that the ddPCR method established by the present invention is correct.

II. Optimization of Conditions for Quantitative Detection of VBNC State Bacteria

1. Optimization of Specificity of Primers

Using bacterial genomic DNA as a template, fluorescent quantitative PCR was performed with different concentrations of rfbE-F and rfbE-R, wherein the final primer concentrations in the system were 200 nmol/L, 300 nmol/L, 400 nmol/L, 500 nmol/L, 600 nmol/L, 700 nmol/L and 800 nmol/L, respectively. The Ct values were compared at different primer concentrations.

The qPCR reaction system was as follows (total volume: 20 μl): 10 μl of 2× SsoFast™ EvaGreen (Bio-Rad, catalog number: 172-5200), 1 μl of forward primer, 1 μl of reverse primer, 1 μl of DNA template, DEPC water was added to a final volume of 20 μl.

The qPCR reaction conditions were as follows: 95° C. for 5 min; 45 cycles of (95° C. for 10 s, 60° C. for 30 s); the fluorescence was collected at 60° C.

The results are shown in Table 1. As can be seen from Table 1, when the primer concentration was 500 nmol/L, the Ct value was the lowest, so the optimal primer concentration was 500 nmol/L.

TABLE 1
Screening of primer concentration

| | Primer concentration (nM) | | | | | | |
|---|---|---|---|---|---|---|---|
| | 200 | 300 | 400 | 500 | 600 | 700 | 800 |
| Ct value | 27.03 | 26.53 | 26.40 | 26.40 | 26.46 | 26.18 | 25.76 |

2. Optimization of Primer Annealing Temperature

Using bacterial genomic DNA as a template, fluorescent quantitative PCR was performed with rfbE-F and rfbE-R at different annealing temperatures, wherein the annealing temperatures were 50° C., 51.3° C., 53.9° C., 60° C., 62.6° C., 66.6° C., 68.8° C., 70° C., respectively. The Ct values at different annealing temperatures were compared.

The qPCR reaction system was as follows (total volume: 20 μl): 10 μl of 2× SsoFast™ EvaGreen, 1μl of forward primer, 1μl of reverse primer, 1μl of DNA template, DEPC water was added to a final volume of 20 μl. The final primer concentration was 200 nmol/L

The qPCR reaction conditions were as follows: 95° C. for 5 min; 45 cycles of (95° C. for 10 s, 60° C. for 30 s); the fluorescence was collected at 60° C.

The results are shown in Table 2. As can be seen from Table 2, when the annealing temperature was 60° C., the Ct value was the lowest, so the optimal annealing temperature was 60° C.

TABLE 2
Screening of primer annealing temperature

| | Annealing temperature (° C.) | | | | | | | |
|---|---|---|---|---|---|---|---|---|
| | 50 | 51.3 | 53.9 | 60 | 62.6 | 66.6 | 68.8 | 70 |
| Ct value | 26.75 | 26.76 | 26.71 | 26.38 | 26.52 | 27.32 | 32.13 | 39.38 |

3. Optimization of PMA Concentration

1 ml of the induced bacteria solution (VBNC state E. coli O157:H7) was subjected to a 1:10 stepwise dilution to obtain a bacteria solution with a concentration of 1×10⁷ CFU/ml, and then 1 ml of the bacteria solution with a concentration of 1×10⁷ CFU/ml was taken, and then the following different amounts of PMA were added, respectively: 2.5 μg, 5 μg, 10 μg, 20 μg and 40 μg, incubated in the dark for 30 min, and each incubation product was illuminated at a distance of 20 cm from a 500 W halogen lamp for 15 min to make the PMA fully react and obtain a PMA-treated sample. The genomic DNA of each PMA-treated sample was extracted, and the copy number of the target gene rfbe in each sample was detected by fluorescence quantitative PCR, and the Ct values of fluorescence quantitative PCR at different concentrations of PMA were compared. qPCR reaction system and reaction conditions were the same as step 2.

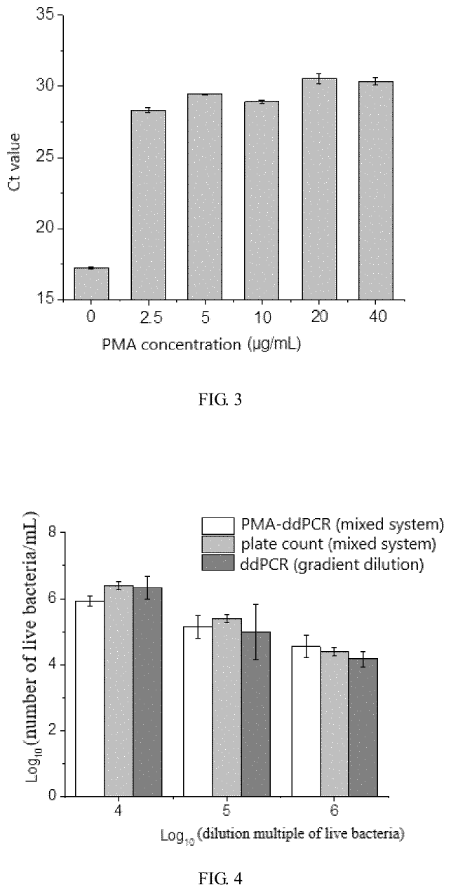

The results are shown in FIG. 3. As can be seen from FIG. 3, when the amount of PMA was 20 μg, the Ct value of quantitative PCR was the highest, indicating the greatest inhibition of dead bacteria, so the optimal amount of PMA was 20 μg.

4. Optimization of Conditions for PMA-ddPCR Detection of Dead/Live Bacteria

1) First, E. coli O157:H7 was cultivated to an OD₆₀₀ of 0.6 (in the logarithmic growth phase) to obtain a bacteria solution with a concentration of 1×10⁸ CFU/ml, and the bacteria solution was subjected to a 1:10 stepwise dilution to obtain live bacteria solutions with concentrations of 1×10⁷ CFU/ml, 1×10⁶ CFU/ml, 1×10⁵ CFU/ml and 1×10⁴ CFU/ml, respectively.

2) 1 ml of the live bacteria solution with a concentration of 1×10⁷ CFU/ml was mixed with 1 ml of 70% (volume fraction) isopropanol solution, lethal for 40min, and a dead bacteria solution with a concentration of 1×10⁷/ml was obtained.

3) Then the live bacteria solutions with concentrations of 1×10⁶ CFU/ml, 1×10⁵ CFU/ml and 1×10⁴ CFU/ml were mixed with the dead bacteria solution with a concentration of 1×10⁷/ml in equal volumes, respectively, and 20 μg of PMA was added to each mixed bacteria solution for PMA treatment (treatment conditions were the same as substep 3 in step I). At the same time, the mixed bacteria solution without PMA treatment was used as a control.

4) The genomic DNA of each PMA-treated mixed bacteria solution was extracted, and the copy number of the target gene rfbe in each PMA-treated mixed bacteria solution was detected by ddPCR (the detection method was the same as substep 5 in step I). At the same time, the same amount of each PMA-treated mixed bacteria solution was taken for plate count (the detection method was the same as substep 2 in step I), and the correlation analysis between the results of copy number detected by ddPCR and the plate count results was performed.

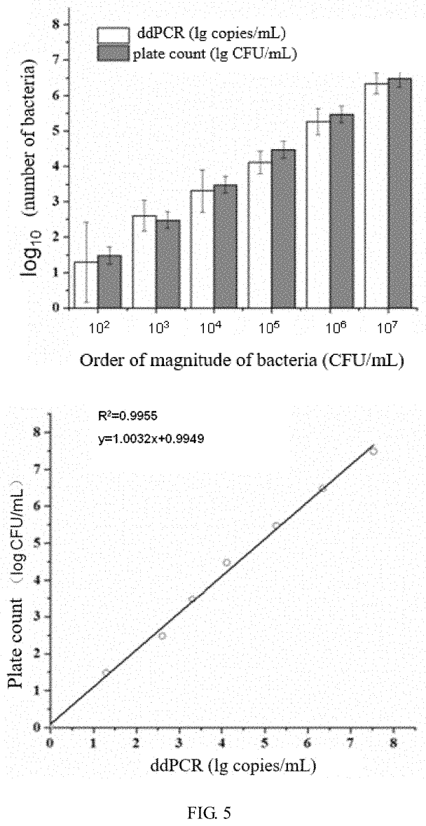

The results are shown in FIG. 4. As can be seen from FIG. 4, when detecting live bacteria in the mixed system of live and dead bacteria by PMA-ddPCR, the results are not significantly different from the plate count results, so PMA-ddPCR can accurately recognize and identify live bacteria in the mixed system.

### EXAMPLE 2

### Correlation Analysis of ddPCR Detection of rfbe Gene Copy Number and Colony Count Method

1. First, E. coli O157:H7 was cultivated to an OD₆₀₀ of 0.6 (in the logarithmic growth phase) to obtain a bacteria solution with a concentration of 1×10⁸ CFU/ml.

2. 1 ml of the bacteria solution with a concentration of 1×10⁸ CFU/ml was subjected to a 1:10 stepwise dilution to obtain live bacteria solutions with concentrations of 1×10⁷ CFU/ml, 1×10⁶ CFU/ml, 1×10⁵ CFU/ml, 1×10⁴ CFU/ml, 1×10³ CFU/ml, 1×10² CFU/ml, and 1×10⁷ CFU/ml, respectively.

3. The genomic DNA of bacteria solutions with different concentrations was extracted respectively, and the copy numbers of the rfbe gene in the samples were detected by ddPCR (the detection method was the same as substep 5 in step I of Example 1). At the same time, the same amount of bacteria solution with different concentrations was taken for plate count (the detection method was the same as substep 2 in step I of Example 1), and the correlation analysis of ddPCR detection of copy number and colony count method was performed.

The results are shown in FIG. 5. As can be seen from FIG. 5, there was no significant difference between the plate count results of each dilution sample and the copy numbers detected by ddPCR, and the two were highly correlated (R²=0.9955), so ddPCR can quantitatively detect bacteria of the order of magnitude of 10¹ CFU.

### EXAMPLE 3

### Detection of Sensitivity of ddPCR

1. First, E. coli O157:H7 was cultivated to an OD₆₀₀ of 0.6 (in the logarithmic growth phase) to obtain a bacteria solution with a concentration of 1×10⁸ CFU/ml.

2. The genomic DNA of the bacteria solution with a concentration of 1×10⁸ CFU/ml was extracted, the concentration of genomic DNA was detected using Bioteke ND5000, and the genomic DNA was subjected to a 1:10 stepwise dilution to obtain genomic DNA samples with DNA contents of 100 ng, 10 ng, 1 ng, 100 pg, 10 pg, 1 pg, 100 fg, 10 fg and 1 fg, respectively.

3. ddPCR

The copy numbers of rfbe gene in the genomic DNA samples with different DNA contents were detected by ddPCR (the detection method was the same as substep 5 in step I of Example 1).

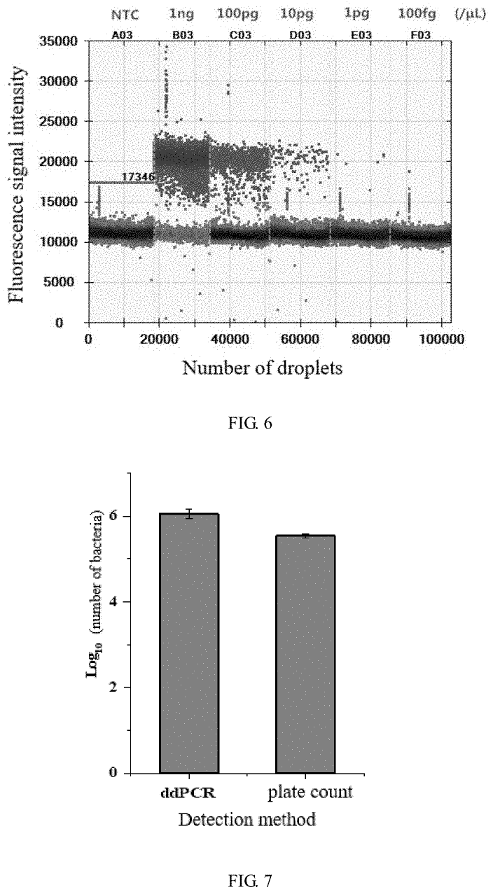

The results are shown in FIG. 6. As can be seen from FIG. 6, the lowest limit of detection of ddPCR was 100 fg, and the copy number of the target gene cannot be detected when the DNA content in the sample was less than 100 fg.

### EXAMPLE 4

### Detection of Specificity of ddPCR

1. Preparation of Bacteria Solution to be Tested

An E. coli O157:H7 solution with a concentration of 1×10⁵ CFU/ml was mixed with a Staphylococcus aureus solution (S. aureus strain was ATCC 6538P, deposit number: CGMCC1.1861) with a concentration of 1×10⁵ CFU/ml, Lactobacillus plantarum solution (Lactobacillus plantarum strain was L. plantarum, deposit number: CGMCC No. 14398) with a concentration of 1×10⁵ CFU/ml, Lactobacillus curvatus solution (Lactobacillus curvatus strain was L. curvatus, deposit number: CGMCC No.14397) with a concentration of 1×10⁵ CFU/ml and Bacillus solution (Bacillus strain was B. subtilis 168, deposit number: CGMCC 1.1088) with a concentration of 1×10⁵ CFU/ml were mixed in equal volumes, respectively, to obtain mixed bacteria solutions.

2. The genomic DNA of the mixed bacteria solutions in step 1 was extracted, respectively, and the specificity of ddPCR method for amplification of target gene rfbe primer was detected. Meanwhile, the number of E. coli O157:H7 was counted using plate count method. The plate count results were compared with the ddPCR detection results.

The results are shown in FIG. 7. ddPCR can detect E. coli O157:H7 with a good specificity, while the amplification numbers of the other four bacteria (Staphylococcus aureus, Lactobacillus plantarum, Lactobacillus curvatus and Bacillus) were all 0 copy/μL and can be ignored. It shows that the ddPCR method established by the present invention has a good specificity.

### INDUSTRIAL APPLICATIONS

The present invention provides a simple and rapid method for quantitatively detecting VBNC state bacteria and applies ddPCR to detect and quantify VBNC state bacteria for the first time. The present invention uses the combination of PMA and ddPCR to distinguish dead and live bacteria in a sample and can accurately identify live bacteria and VBNC state bacteria in the sample and uses Dead/Live staining combined with flow cytometry to verify the detection results of ddPCR. It is proved by experiments that the detection method of the present invention can achieve accurate detection and quantification of VBNC state bacteria within 4-6 h with a detection range of 10¹-10⁷ and a quantitative range of 10²-10⁷. This method not only has the advantages of strong specificity and high sensitivity, but also has the advantages of accurate quantification, reliable results, simplicity and time saving. The PMA-ddPCR method of the present invention can accurately quantify the amount of VBNC state bacteria that may be present in the food during the food processing, and can more comprehensively and accurately carry out their pathogenic risk assessment. The present invention is of great significance both for the detection and quantification of VBNC state bacteria in food and for the management and monitoring of food safety.

What is claimed is:

1. A method for quantitatively detecting VBNC state bacteria, comprising the following steps:
1) treating the VBNC state bacteria to be tested with propidium monoazide to obtain propidium monoazide-treated bacteria;
2) performing ddPCR amplification on a target gene in the VBNC state bacteria to be tested with the genomic DNA of the propidium monoazide-treated bacteria as a template to obtain a copy number of the target gene;
3) determining the number of the VBNC state bacteria to be tested according to the copy number of the target gene.

2. The method according to claim 1, wherein the method for treating the VBNC state bacteria to be tested with propidium monoazide comprises the following steps: mixing the bacteria solution of the VBNC state bacteria to be tested with propidium monoazide and incubating the resulting mixture to obtain an incubation product; subjecting the incubation product to light treatment to obtain the propidium monoazide-treated bacteria.

3. The method according to claim 2, wherein the ratio of the VBNC state bacteria to be tested to propidium monoazide is 1×10⁷ CFU:(15-23) μg.

4. The method according to claim 2, wherein the incubation condition is 30° C. for 15-30 min.

5. The method according to claim 2, wherein the light treatment is illuminating the incubation product at a distance of 20 cm from a 500 W halogen lamp for 10-20 min.

6. The method according to claim 1, wherein the bacteria are E. coli strains.

7. The method according to claim 6, wherein the E. coli strains are E. coli O157:H7 strains.

8. The method according to claim 7, wherein the target gene in the E. coli O157:H7 strains is rfbe gene.

9. The method according to claim 8, wherein the primer pair used for the ddPCR amplification of the rfbe gene consists of the single-stranded DNA molecule set forth in SEQ ID NO: 1 and the single-stranded DNA molecule set forth in SEQ ID NO: 2.

10. The method according to claim 9, wherein the final concentration of each primer in the primer pair in ddPCR amplification reaction system is 500 nmol/L.

11. The method according to claim 9, wherein the annealing temperature of the ddPCR amplification of the rfbe gene is 60° C.

12-13. (canceled)

14. A method for quantitatively detecting VBNC state bacteria in a sample to be tested, comprising the following steps:
1) treating the sample to be tested with propidium monoazide to obtain a propidium monoazide-treated sample;
2) performing ddPCR amplification on a target gene in the VBNC state bacteria in the sample to be tested with the genomic DNA of the propidium monoazide-treated sample as a template to obtain a copy number of the target gene;
3) determining the number of the VBNC state bacteria in the sample to be tested according to the copy number of the target gene.

15. The method according to claim 14, wherein the method for treating the sample to be tested with propidium monoazide comprises the following steps: mixing the sample to be tested with propidium monoazide and incubating the resulting mixture to obtain an incubation product; subjecting the incubation product to light treatment to obtain the propidium monoazide-treated sample.

16. The method according to claim 15, wherein the incubation condition is 30° C. for 15-30 min.

17. The method according to claim 15, wherein the light treatment is illuminating the incubation product at a distance of 20 cm from a 500 W halogen lamp for 10-20 min.

18. A kit for quantitative detection of VBNC state bacteria, comprising propidium monoazide and a primer pair used for ddPCR amplification of a target gene in the bacteria.

19. The kit according to claim 18, wherein the bacteria are E. coli strains.

20. The kit according to claim 18, wherein the E. coli strains are E. coli O157:H7 strains.

21. The kit according to claim 18, wherein the target gene in the E. coli O157:H7 strains is rfbe gene.

22. The kit according to claim 21, wherein the primer pair used for the ddPCR amplification of the rfbe gene consists of the single-stranded DNA molecule set forth in SEQ ID NO: 1 and the single-stranded DNA molecule set forth in SEQ ID NO: 2.
